SEQUENCE LISTING (2 sequences)

SEQ ID NO: 1 - DNA, 20 bp, synthetic construct
  source 1..20: mol_type = other DNA
agagtttgatcctggctcag

SEQ ID NO: 2 - DNA, 20 bp, synthetic construct
  source 1..20: mol_type = other DNA
ctacggctaccttgttacga

(19) United States
(12) Patent Application Publication
(10) Pub. No.: US 2026/0273001 A1
(43) Pub. Date: Sep. 17, 2026

(54) PROBIOTIC STRAIN WITH EFFICACY IN IMPROVING DEPRESSION-LIKE SYMPTOMS, MICROECOLOGICAL PREPARATION AND USES THEREOF

(71) Applicant: Ningbo University, Ningbo (CN)

(72) Inventors: Xiaoqun ZENG, Ningbo (CN); Ling LI, Ningbo (CN); Daodong PAN, Ningbo (CN); Shufeng HE, Ningbo (CN); Zhen WU, Ningbo (CN); Qiwei DU, Ningbo (CN); Yan TENG, Ningbo (CN); Tao ZHANG, Ningbo (CN)

(21) Appl. No.: 19/671,398

(22) Filed: May 8, 2026

(30) Foreign Application Priority Data
Jun. 18, 2025 (CN) 202510813731.6

(51) Int. Cl. A61K 35/747; A61P 25/24; C12N 1/20; C12R 1/225

(52) U.S. Cl. CPC A61K 35/747; A61P 25/24; C12N 1/20; C12R 2001/225

(57) ABSTRACT

The present disclosure discloses a probiotic strain with efficacy in improving depression-like symptoms, a microecological preparation and uses thereof. The probiotic strain is Limosilactobacillus fermentum PC17, which has been deposited under the culture collection number CGMCC No. 33866, the active ingredient of the lactic acid bacteria microecological preparation comprise Limosilactobacillus fermentum PC17. The microecological preparation is prepared by inoculating Limosilactobacillus fermentum PC17 into a fermentation medium at 1-5% (v/v) to obtain a probiotic microecological preparation. The preparation can be used for the manufacture of antidepressant drugs. The advantages include: promoting the secretion of 5-HT and 5-HTP by RIN14B cells, reducing serum levels of IL-1β, IL-6, TNF-α, CORT, and ACTH, and increasing serum levels of GABA and BDNF. Furthermore, the depression-like behaviors in a CUMS-induced depression-like mouse model are significantly ameliorated.

### SEQUENCE LISTING

The Sequence Listing XML file named “Sequence_Listing.xml,” created on Jun. 16, 2025, and having a size of 2,737 bytes, contains the sequence listing for this application and is hereby incorporated by reference in its entirety.

### TECHNICAL FIELD

The present disclosure relates to lactic acid bacteria, particularly a probiotic strain with efficacy in improving depression-like symptoms, a microecological preparation and uses thereof.

### BACKGROUND

Depression is a highly prevalent psychiatric disorder worldwide, characterized by persistent low mood, loss of interest, psychomotor retardation, and pessimistic ideation as its primary clinical symptoms. The illness is characterized by recurrent episodes of long duration and is associated with high morbidity, mortality, and suicide rates, posing a serious threat to human physical and mental health. With the rapid pace of modern society and increasing life and work stress, the incidence of depression has been rising. According to the World Health Organization, approximately 3.8% of the global population suffers from depression, with around 700,000 suicide deaths annually attributable to depression, making it a mental disorder with a major contributor to global disease burden and disability. The pathogenesis of depression involves a complex interplay of multiple factors, including monoamine neurotransmitter imbalance, hyperactivity of the hypothalamic-pituitary-adrenal (HPA) axis, and chronic inflammatory responses. While conventional antidepressant drugs demonstrate some efficacy, they are limited by delayed onset of action, significant side effects, and substantial inter-individual variability. Therefore, there remains a need to develop novel antidepressant therapies.

In recent years, the microbiota-gut-brain axis theory has provided a new perspective for the treatment of depression. Specifically, the gut microbiota can affect the function of the central nervous system directly or indirectly through metabolites and immune regulation. Consequently, probiotics have emerged as a research interest for mental disorder intervention due to their potential to modulate gut microbiota homeostasis and neuroimmune function. Accordingly, the development of probiotics with efficacy in alleviating depression and their application in alleviating depression holds significant research importance.

### SUMMARY

The problem addressed by the present disclosure is to provide a probiotic strain with efficacy in improving depression-like symptoms, a microecological preparation and uses thereof, wherein the probiotic strain promotes the secretion of 5-hydroxytryptamine (5-HT) and 5-hydroxytryptophan (5-HTP) in RIN14B cells, reduces serum levels of interleukin-16 (IL-1β), interleukin-6 (IL-6), tumor necrosis factor-α (TNF-α), corticosterone (CORT), and adrenocorticotropic hormone (ACTH), and increases serum levels of gamma-aminobutyric acid (GABA) and brain-derived neurotrophic factor (BDNF).

The technical solution adopted by the present disclosure to solve the above technical problems is as follows:

The present disclosure provides a probiotic strain with efficacy in improving depression-like symptoms, the probiotic strain is Limosilactobacillus fermentum PC17, deposited under culture collection number CGMCC No. 33866.

The present disclosure further provides a probiotic microecological preparation with efficacy in improving depression-like symptoms, including Limosilactobacillus fermentum PC17, the strain having a culture collection number CGMCC No. 33866, active ingredients of a lactic acid bacteria microecological preparation include the bacteria of Limosilactobacillus fermentum PC17.

Further, an effective viable cell count of Limosilactobacillus fermentum PC17 in the probiotic microecological preparation is greater than or equal to 1×10⁷ CFU/mL.

The present disclosure further provides a method for preparing the probiotic microecological preparation with efficacy in improving depression-like symptoms, including the following steps: inoculating Limosilactobacillus fermentum PC17, deposited under the culture collection number CGMCC No. 33866, into a fermentation medium at 1-3% (v/v) and culturing at 35-40° C. for 15-20 h to obtain a lactic acid bacterial culture; centrifuging the obtained lactic acid bacterial culture to obtain a lactic acid bacterial pellet, adjusting a bacterial concentration to 1×10⁷-1×10⁹ CFU/mL using sterile phosphate-buffered saline (PBS); adding a lyoprotectant in an amount equal to the sterile PBS and mixing uniformly, then placing the mixture in a refrigerator at −80° C. overnight for pre-freezing, and freeze-drying the pre-frozen sample to obtain the probiotic microecological preparation.

Further, the lyoprotectant includes 118.2 g/L trehalose, 17.1 g/L L-cysteine, 10.3 g/L sorbitol, 1.7 g/L sodium acetate and 120 g/L skimmed milk powder.

The present disclosure further provides a use for the above-mentioned probiotics in the preparation of an antidepressant composition.

Compared with the prior art, the present disclosure has the following advantages: it is the first time to disclose a probiotic strain with efficacy in improving depression-like symptoms and uses thereof, whereby the serum levels of IL-1β, IL-6, TNF-α, CORT, and ACTH in mice are effectively reduced, and the serum levels of GABA and BDNF in mice are increased. Furthermore, depression-like behaviors in a chronic unpredictable mild stress (CUMS)-induced depression mouse model are significantly ameliorated. Thus, the present disclosure provides a novel approach in functional products or compositions for depression relief and overcomes the limitations of conventional antidepressant drugs, such as slow onset of action and significant side effects.

Limosilactobacillus fermentum PC17, with a culture collection number CGMCC No. 33866, is deposited at the China General Microbiological Culture Collection Center and Management Committee on Mar. 19, 2025, at No. 3, No. 1 West Beichen Road, Chaoyang District, Beijing, China.

### BRIEF DESCRIPTION OF THE DRAWINGS

FIG. 1 illustrates lactic acid bacteria stimulating secretion of 5-HT from RIN14B cells;

FIG. 2 illustrates lactic acid bacteria stimulating secretion of 5-HTP from RIN14B cells;

FIG. 3 illustrates a self-aggregation ability of QD2 and PC17;

FIG. 4 illustrates hydroxyl radical scavenging rates for QD2 and PC17;

FIG. 5 illustrates 1,1-diphenyl-2-picrylhydrazyl (DPPH) radical scavenging rates of QD2 and PC17;

FIG. 6 illustrates a growth curve of QD2 and PC17;

FIG. 7 illustrates a phylogenetic tree of PC17;

FIG. 8 illustrates experimental results of sugar water preference in mice;

FIG. 9 illustrates experimental results of forced swimming in mice;

FIG. 10 illustrates experimental results of a tail suspension test in mice;

FIG. 11 illustrates serum 5-HT levels in mice;

FIG. 12 illustrates serum 5-HTP levels in mice;

FIG. 13 illustrates serum GABA levels in mice;

FIG. 14 illustrates serum IL-1β levels in mice;

FIG. 15 illustrates serum IL-6 levels in mice;

FIG. 16 illustrates serum TNF-α levels in mice;

FIG. 17 illustrates serum ACTH levels in mice;

FIG. 18 illustrates serum CORT levels in mice;

FIG. 19 illustrates serum BDNF levels in mice;

FIG. 20 illustrates a hematoxylin and eosin (HE) staining of mouse jejunum tissue;

FIG. 21 illustrates an effect of lactic acid bacteria microecological preparations on species relative abundances at a phylum level of intestinal bacteria;

FIG. 22 illustrates an effect of lactic acid bacteria microecological preparations on species relative abundances at a genus level of intestinal bacteria;

Note: Group C: blank group; Group H: CUMS model group; fluoxetine hydrochloride (FLX) group: positive control fluoxetine hydrochloride group; PC17-H: PC17 high dose group; PC17-M: PC17 medium dose group; PC17-L: PC17 low dose group, compared with Group C *P<0.05, **P<0.01, ***P<0.001, ****P<0.0001.

### DETAILED DESCRIPTION

The following describes the present disclosure in further detail with reference to the accompanying drawings.

### I. Experimental Method

### 1. Medium and Solution Preparation

Cell complete medium: 9% fetal bovine serum (FBS), 90% Dulbecco's Modified Eagle Medium (DMEM), and 1% penicillin-streptomycin.

Acid-resistant screening medium: The pH of the MRS broth medium is adjusted to 2.5, 3.5, and 4.5 with hydrochloric acid (HCl).

Bile salt-tolerance screening medium: Pig bile salt is added to MRS broth medium at concentrations of 0.3 g/L and 0.6 g/L, respectively.

HBSS buffer: 0.1% fetal bovine serum (FBS) and 2 μM fluoxetine hydrochloride are added to the HBSS solution.

### 2. Activation and Preservation of Strains

2 mL of the strain preserved in the refrigerator at −80° C. is obtained and inoculated into MRS broth medium, followed by culturing at 37° C. for 12 h to obtain a seed liquid. Then the seed liquid is diluted, spread onto MRS agar medium, and after observation of colony morphology, the plates are placed in the refrigerator at 4° C. for future use.

### 3. Determination of Acid Resistance

The activated lactic acid bacteria are inoculated into MRS liquid medium with pH 2.5, 3.5 and 4.5 at a 3% volume fraction, followed by incubation at 37° C. for 16 h. Subsequently, the OD₆₀₀ value of each group is measured.

### 4. Determination of Bile Salt Tolerance

The activated lactic acid bacteria are inoculated into MRS liquid medium with bile salt concentrations of 0 g/L, 0.3 g/L, and 0.6 g/L at 3% volume fraction, and cultured anaerobically at 37° C. for 16 h. The OD_{600 nm} value of each group is measured.

### 5. Preparation of the Lactic Acid Bacteria Liquid

The acid-tolerant and bile salt-tolerant lactic acid bacteria seed liquid is inoculated into MRS broth medium and cultured at 37° C. for 24 h until the late logarithmic growth phase is reached. This activation process is repeated for three consecutive generations to obtain a lactic acid bacterial culture. Then the culture is centrifuged at 4000 rpm and 4° C. for 5 min, and the supernatant is removed. The bacterial pellet is washed twice with sterile PBS, resuspended, and subjected to viable cell counting using a 10-fold serial dilution method. Then the bacterial concentration is adjusted to 1×10⁸ CFU/mL before cell stimulation.

### 6. Co-Culture of Lactic Acid Bacteria and Cells

RIN14B cells are seeded in 24-well plates at a density of 4×10⁶ cells/mL. After incubation for 72 h, the medium is discarded, and the cells are washed with 1 mL of HBSS buffer (2-3 times). Subsequently, 1 mL of HBSS suspension containing viable bacteria at a concentration of 1×10⁸ CFU/mL is added (the control group is added HBSS only), and the plates are incubated at 37° C. for 30 min. Then the supernatant is collected and centrifuged at 4° C. and 6000-10000×g for 5 min to remove the pellet. The resulting supernatant is collected and frozen at −80° C. for future testing.

### 7. Determination of Self-Aggregation Ability

The bacterial suspension to be tested is centrifuged at 10,000 r/min for 3 min at 4° C. The supernatant is removed, and the bacterial pellet is collected. The bacterial pellet is washed twice with PBS, and the bacterial concentration is adjusted with buffer to obtain a bacterial suspension having an absorbance value of approximately 1.000 (0.8±0.1) at a wavelength of 600 nm. Then the lactic acid bacteria suspension is placed at 37° C. for 4 h and 24 h, respectively. The absorbance of the supernatant is measured at 600 nm at 0 h, 4 h, and 24 h. The self-aggregation rate is calculated using the following formula:

$$self - aggregation ( \% ) = ( 1 - \frac{{A}_{i}}{{A}_{c}} ) \times 100 \%$$

where Aᵢ is the absorbance at 4 h or 24 h of treatment; A_{c} is the initial absorbance of the lactic acid bacteria suspension.

### 8. Determination of the Ability to Scavenge Hydroxyl Radicals (OH)

A mixture is prepared by adding 1 mL of 2 mmol/L FeSO₄ solution, 1 mL of 6 mmol/L H₂O₂, 1 mL of 6 mmol/L salicylic acid, and 1 mL of bacterial culture supernatant. The mixture is allowed to stand for 30 min at room temperature, with deionized water serving as the blank control. Subsequently, the absorbance at 510 nm is measured, and the hydroxyl radical scavenging rate is calculated.

$$hydroxyl radical scavenging rate ( \% ) = ( 1 - \frac{{A}_{i}}{{A}_{c}} ) \times 100 \%$$

where Aᵢ is the absorbance of the treatment group, and A_{c} is the absorbance of the blank control group.

### 9. Determination of DPPH Radical Scavenging Ability

A 0.2 mmol/L DPPH anhydrous ethanol solution is prepared by dissolving 19.72 mg DPPH in 250 mL anhydrous ethanol. 2 mL of the sample is mixed with 2 mL of the DPPH anhydrous ethanol solution, and the mixture is placed at room temperature for 30 min in the dark, followed by centrifugation at 6000 rpm for 10 min. Then the absorbance of the supernatant is measured at 517 nm. For the blank group, the DPPH anhydrous ethanol solution is replaced with an equal volume of anhydrous ethanol. For the control group, the sample solution is replaced with distilled water.

$$DPPH radical scavenging rate ( \% ) = ( 1 - \frac{{A}_{i} - {A}_{c}}{{A}_{j}} ) \times 100 \%$$

where Aᵢ is the absorbance of the treatment group, A_{c} is the absorbance of the blank group, and Aⱼ is the absorbance of the control group.

### 10. Antibiotic Susceptibility Experiment

Approximately 100 μL of the bacterial suspension (prepared with sterile water at a concentration of about 1×10⁸ CFU/mL) is uniformly spread onto MRS medium. Antibiotic susceptibility testing disks are placed onto the surface of the medium using sterile forceps, and each disk is gently pressed to ensure firm contact. The plates are incubated in a constant temperature incubator at 37° C. for 24 h, after which the diameters of the inhibition zones are measured. The experimental results are determined according to the standards of the Clinical and Laboratory Standards Institute (CLSI).

### 11. Determination of Growth Curve

After the seed liquid is activated for three consecutive generations, it is inoculated into the medium at an inoculum volume of 3% (v/v) and cultured. The absorbance of the fermentation broth at OD₆₀₀ is measured every 2 h. Before each measurement, the culture medium is fully shaken, and three replicate measurements are performed.

12. Identification and Phylogenetic Analysis of 16S rDNA of Lactic Acid Bacteria

### (1) DNA Extraction

The fresh bacterial liquid is centrifuged at 8000 rpm for 1 min to obtain the bacteria, and the total DNA of the strain is extracted with a kit.

(2) the 16S rRNA Gene is Amplified by Polymerase Chain Reaction (PCR) Using Universal Primers. The Primer Sequences are as Follows:

Upstream primer 27F:
5′-AGAGTTTGATCCTGGCTCAG-3′.
The downstream primer 1495R:
5′-CTACGGCTACCTTGTTACGA-3′.

The PCR cycling parameters are as follows: pre-denaturation at 94° C. for 5 min; denaturation at 94° C. for 1 min; annealing at 64° C. for 1 min; extension at 72° C. for 2 min; and a final extension at 4° C. for 10 min. A total of 34 cycles is performed. Then the amplified PCR products are subjected to nucleic acid electrophoresis, and the products are sent to Sangon Biotech Co., Ltd. for gene sequence determination.

### (3) Phylogenetic Tree

The obtained strain sequence is aligned with the 16S rDNA gene fragment on the National Center for Biotechnology Information (NCBI) website, and the 16S rDNA gene sequence of the strain with high similarity is extracted, and the phylogenetic tree is constructed using the software MEGA X 64.

### (4) Data Processing and Statistical Analysis

Each experiment is repeated three times. Statistical analysis of the experimental data and the plotting of graphs are performed using GraphPad Prism 9.4.0 and SPSS 19.0. The experimental results are presented in the form of mean±standard deviation (mean±SEM). Significance tests for data from multiple groups are performed using one-way analysis of variance (one-way ANOVA).

### II. Screening of Strains

### 1. Screening of Lactic Acid Bacteria

160 strains of lactic acid bacteria are randomly selected from the strain library of the College of Food Science and Engineering of Ningbo University for acid and bile salt tolerance screening, and 15 strains of lactic acid bacteria with good acid and bile salt tolerance are obtained. The results are shown in Table 1.

TABLE 1
Source and collection site of lactic acid bacteria

| No. | Isolation Habitat | Collection site |
|---|---|---|
| 63-KG-31 | Fermented vinegar grains | Yantai, Shandong |
| QD1 | Pickled vegetables | Dongtou, Zhejiang |
| QD8 | Pickled vegetables | Dongtou, Zhejiang |
| QD13 | Pickled vegetables | Dongtou, Zhejiang |
| PC17 | Pickled vegetables | Dongtou, Zhejiang |
| QD18 | Pickled vegetables | Dongtou, Zhejiang |
| VIC2 | Pickled radish | Hangzhou, Zhejiang |
| DFHS | Pickled wax gourd | Hangzhou, Zhejiang |
| MS2 | Intestinal samples from boys | Ningbo, Zhejiang |
| MS3 | Intestinal samples from boys | Ningbo, Zhejiang |
| QD2 | Pickled vegetables | Dongtou, Zhejiang |
| QD11 | Pickled vegetables | Dongtou, Zhejiang |
| QD14 | Pickled vegetables | Dongtou, Zhejiang |
| 13-KC-35 | Pickled water bamboo | Ningbo, Zhejiang |
| | shoots | |
| 64-DM-7 | Traditional fermented yogurt | Hohhot, Inner Mongolia |

### 2. Determination of the Ability of Lactic Acid Bacteria to Stimulate 5-HT/5-HTP Production in Cells

Numerous studies have demonstrated that depression is associated with dysregulation of monoamine neurotransmitters in the brain, primarily manifested by a decrease in the concentration of 5-HT in the synaptic cleft. Currently, selective serotonin reuptake inhibitors (SSRIs) are the main drugs used for antidepressant treatment. Their primary mechanism of action involves selectively inhibiting the reuptake of 5-HT by acting on the 5-HT transporter on the presynaptic membrane, thereby increasing synaptic 5-HT concentrations to achieve an antidepressant effect. 5-HTP, a precursor for 5-HT synthesis, is rapidly metabolized into 5-HT upon entering the brain, thus safely increasing brain 5-HT levels and alleviating depression and other 5-HT deficiency-related disorders.

In vivo, approximately 90% of 5-HT is synthesized by enterochromaffin (EC) cells; however, EC cells are difficult to isolate and culture. RIN14B cells (ATCC® CRL-2059), an insulinoma cell line, have been demonstrated to serve as a suitable in vitro substitute for EC cells in screening for lactic acid bacteria with high 5-HT-producing capacity.

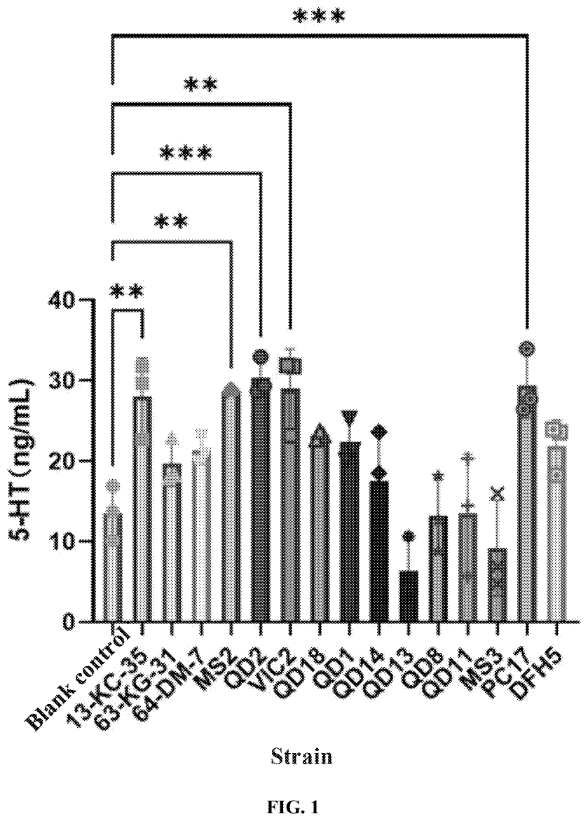

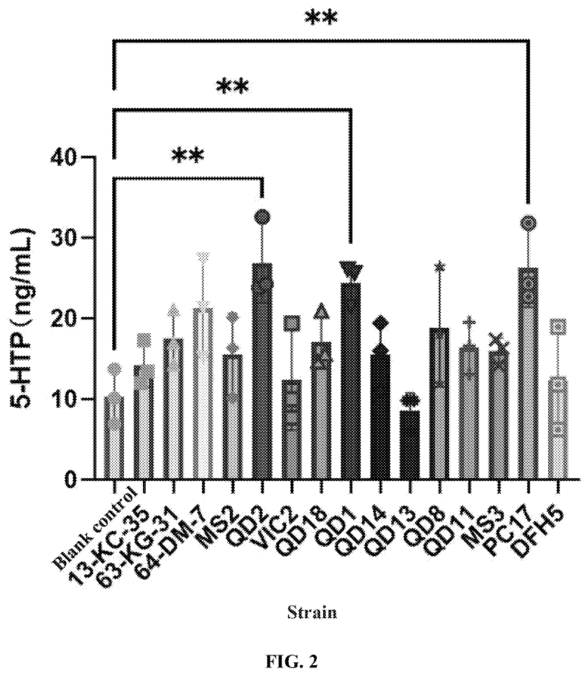

The effects of 15 probiotic strains on 5-HT and 5-HTP secretion in RIN14B cells are evaluated following a 30-minute co-incubation period involving RIN14B cells and lactic acid bacteria. The experimental results are shown in FIGS. 1 and 2: strains MS2 (28.79 ng/mL), PC17 (28.75 ng/mL), 13-KC-35 (27.65 ng/ml), VIC2 (28.98 ng/mL), and QD2 (29.36 ng/mL) are found to significantly stimulate 5-HT secretion in RIN14B cells. Additionally, strains QD2 (26.92 ng/mL), QD1 (24.41 ng/mL), and PC17 (26.29 ng/ml) significantly increase 5-HTP levels in RIN14B cells.

3. The determination of self-aggregation ability: Self-aggregation refers to the phenomenon by which bacterial cells spontaneously gather into clusters. Lactic acid bacteria with strong self-aggregation ability are capable of forming microcolonies or biofilm structures through cell-cell interactions, which facilitates their colonization and retention on the intestinal mucosal surface, thereby enabling them to better exert their probiotic functions. Additionally, lactic acid bacteria with strong self-aggregation ability can prevent the adhesion and colonization of pathogenic bacteria on the intestinal mucosa through physical occupancy and competitive exclusion, thereby reducing the risk of pathogenic infection. Self-aggregation rates are generally categorized as follows: 16-35% indicates low ability, 36-50% indicates medium ability, and ≥51% indicates high ability.

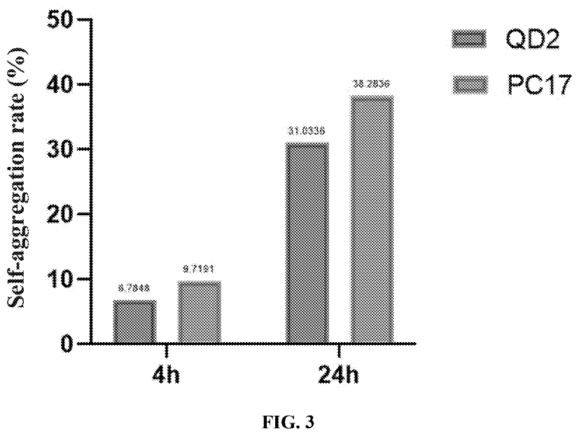

As shown in FIG. 3, the self-aggregation rates of QD2 and PC17 are 6.78% and 9.72% at 4 h, and 31.03% and 38.28% at 24 h, respectively. Based on the 24 h experimental results, QD2 falls into the low self-aggregation category, while PC17 falls into the medium self-aggregation category.

### 4. Determination of the Ability to Scavenge Hydroxyl Radicals (OH)

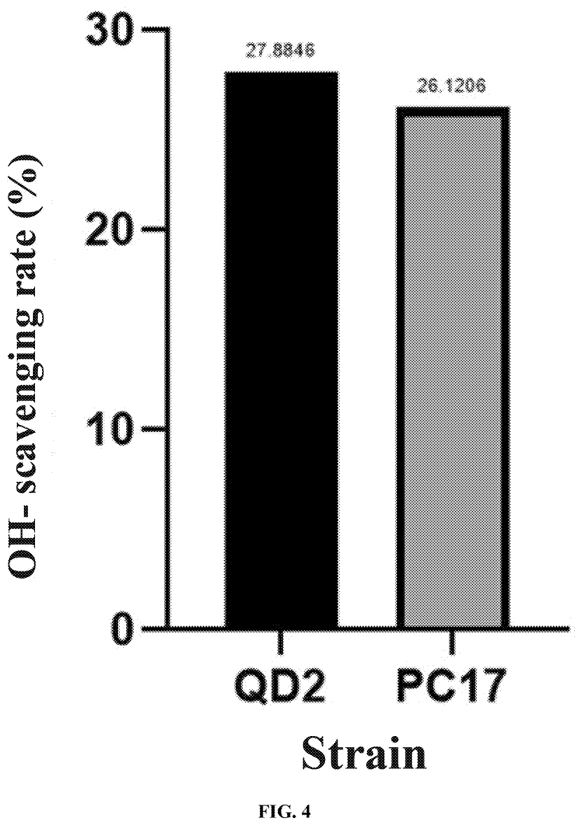

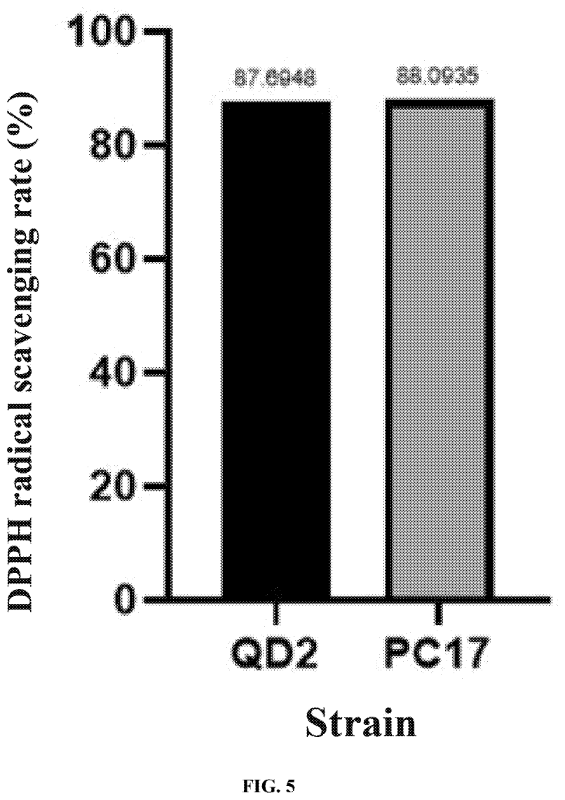

The hydroxyl radical is the most reactive radical in vivo. Due to its high reactivity, it can induce lipid peroxidation, protein denaturation, and DNA damage, thereby triggering a series of harmful biochemical reactions. The ability of lactic acid bacteria to scavenge hydroxyl radicals is an important indicator of their antioxidant function. This ability helps maintain intestinal redox balance and protect host cells from oxidative stress damage. Furthermore, strains with strong antioxidant capacity are more likely to survive and exert probiotic effects within the gastrointestinal environment. The hydroxyl radical scavenging abilities of QD2 and PC17 are determined, and the results are shown in FIG. 4. The hydroxyl radical scavenging rates of QD2 and PC17 are 27.88% and 26.12%, respectively.

### 5. Determination of DPPH Radical Scavenging Ability

DPPH is a synthetic stable radical. The ability of lactic acid bacteria to scavenge DPPH radicals directly reflects their antioxidant activity and serves as an important probiotic function index for strain screening. The DPPH radical scavenging abilities of QD2 and PC17 are evaluated, and the results show that the scavenging rates of QD2 and PC17 are 87.69% and 88.09%, respectively.

### 6. Determination of Antibiotic Susceptibility

As a probiotic widely used in the food industry and healthcare, the antibiotic susceptibility of lactic acid bacteria is an important indicator for evaluating strain safety. Antibiotic susceptibility testing allows for the assessment of whether the strain carries transferable antibiotic resistance genes, thereby preventing the horizontal transfer of resistance genes within the intestinal microbiota and ensuring the safety of its application.

The drug resistance experiment is performed on strains QD2 and PC17. Nine antibiotic susceptibility disks are selected, including ceftazidime, tetracycline, doxycycline, penicillin, erythromycin, streptomycin, gentamicin, minocycline, and ampicillin. The results are shown in Table 2. Based on the diameters of the inhibition zones, both strains are susceptible to all nine antibiotics tested.

TABLE 2
Antibiotic susceptibility of lactic acid bacteria

| | | | | | QD2 | | PC17 | |
|---|---|---|---|---|---|---|---|---|
| | Disk | Interpretation Criteria (mm) | | | Inhibition | | Inhibition | |
| | Potency | Resistant | Intermediate | Susceptible | Zone Diameter | | Zone Diameter | |
| Antibiotic | (μg/disk) | (R) | (I) | (S) | (mm) | | (mm) | |
| Ceftazidime | 30 | ≤14 | 15-17 | ≥18 | 33 ± 0 | (S) | 33 ± 1 | (S) |
| Tetracycline | 30 | ≤14 | 15-18 | ≥19 | 27 ± 2 | (S) | 27.33 ± 0.58 | (S) |
| Doxycycline | 30 | ≤14 | 15-18 | ≥19 | 28.67 ± 1.53 | (S) | 29.33 ± 1.15 | (S) |
| Penicillin | 10 | ≤17 | 18-20 | ≥21 | 42.33 ± 0.58 | (S) | 44.33 ± 3.21 | (S) |
| Erythromycin | 15 | ≤13 | 14-22 | ≥23 | 34 ± 0 | (S) | 34.67 ± 0.58 | (S) |
| Streptomycin | 10 | ≤14 | 15-17 | ≥18 | 10.67 ± 0.58 | (S) | 12.67 ± 0.58 | (S) |
| Gentamicin | 10 | ≤12 | 13-14 | ≥15 | 17 ± 1 | (S) | 17.33 ± 0.58 | (S) |
| Minocycline | 30 | ≤12 | 13-17 | ≥18 | 35.33 ± 2.31 | (S) | 36.33 ± 0.58 | (S) |
| Ampicillin | 10 | ≤13 | 14-16 | ≥17 | 44.67 ± 2.08 | (S) | 48 ± 0 | (S) |

### 7. Growth Curve

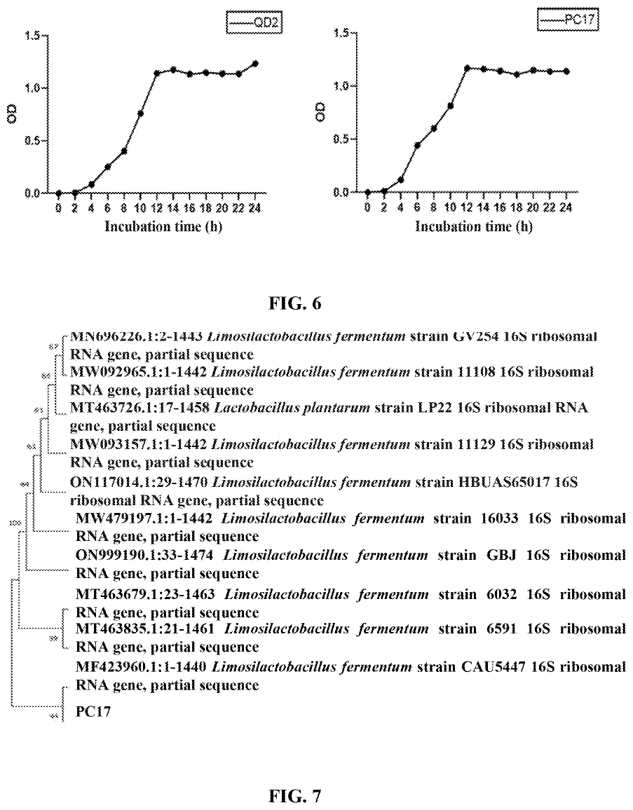

The growth of the strain at different stages can be visually observed by the growth curve. The growth curves of QD2 and PC17 are shown in FIG. 6. Both strains enter the logarithmic growth phase at 3 h and stationary phase after 12 h.

### 8. Phylogenetic Tree and Homology

The phylogenetic tree of strain PC17 is shown in FIG. 7. It can be seen that strain PC 17 is Limosilactobacillus fermentum.

In view of the foregoing, 160 strains of lactic acid bacteria are randomly selected from the strain library of the College of Food Science and Engineering of Ningbo University for acid and bile salt tolerance screening, and 15 strains of lactic acid bacteria with good acid and bile salt tolerance are obtained. Subsequently, the RIN-14B cell model is employed to evaluate the ability of these 15 strains to stimulate the production of 5-HT and its precursor 5-HTP following co-incubation with the cells. Based on this evaluation, QD2 and PC17 are screened as strains with high 5-HT and 5-HTP production. On this basis, multiple functional properties of QD2 and PC17 are evaluated, including antibiotic susceptibility, self-aggregation ability, hydroxyl radical scavenging ability, and DPPH radical scavenging ability. The excellent antioxidant and safety properties of PC17 are determined.

The above-mentioned Limosilactobacillus fermentum PC17 with a culture collection number CGMCC No. 33866, is deposited at the China General Microbiological Culture Collection Center and Management Committee on Mar. 19, 2025, at No. 3, No. 1 West Beichen Road, Chaoyang District, Beijing, China.

### III. Product Application Embodiment

### SPECIFIC EMBODIMENTS

### Embodiment

A probiotic strain with efficacy in improving depression-like symptoms is identified and named as Limosilactobacillus fermentum PC17. The strain is deposited at the China General Microbiological Culture Collection Center and Management Committee on Mar. 19, 2025 under culture collection number CGMCC No. 33866.

### Embodiment 2

A method for preparing the probiotic microecological preparation with efficacy in improving depression-like symptoms, including the following steps:

(1) Lactic acid bacteria fermentation: Limosilactobacillus fermentum PC17 is inoculated into a fermentation medium at a volume ratio of 3% and cultured at 37° C. for 18 h to obtain a lactic acid bacterial culture.

(2) Freeze-drying: The obtained lactic acid bacterial culture is centrifuged at 8000 rpm and 4° C. for 15 min. The bacterial pellet is collected, and the cell concentration is adjusted to 1×10⁸ CFU/mL using sterile PBS. The lyoprotectant in an amount equal to the volume of the sterile PBS is added and mixed uniformly, then the mixture is placed in a refrigerator at −80° C. overnight for pre-freezing. Then the pre-frozen sample is freeze-dried at −49° C. and 9 Pa for 48 h to obtain a lactic acid bacteria composite microecological preparation. The lyoprotectant includes the following components: 118.2 g/L trehalose, 17.1 g/L L-cysteine, 10.3 g/L sorbitol, 1.7 g/L sodium acetate, and 120 g/L skimmed milk powder.

(3) Administration method: The preparation is administered by oral gavage at a cell concentration of 1×10° C. FU/mL, and is designated as the PC17 low dose group (PC17-L).

### Embodiment 3

As in Embodiment 2 described above, the difference is that the bacterial concentration administered by oral gavage in step (3) is 1×10⁹ CFU/mL; this is designated as the PC17 medium dose group (PC17-M).

### Embodiment 4

As in Embodiment 2 described above, the difference is that the bacterial concentration administered by oral gavage in step (3) is 1×10¹⁰ CFU/mL; this is designated as the PC17 high dose group (PC17-H).

### 2. The Alleviating Effect of Lactic Acid Bacteria Probiotics on Depressive Symptoms in CUMS Model Mice

1) Experimental materials: 60 SPF C57BL/6N mice, male, weighing 18-22 g and aged 6-8 weeks, are provided by Zhejiang Vital River Laboratory Animal Technology Co., Ltd. with a production license No. SCXK (Zhejiang) 2019-0001. The animals are housed in a barrier environment with a license No. SYXK (Zhejiang) 2024-0002.

2) Experimental protocol: After one week of acclimatization, the 60 mice are weighed and randomly divided into six groups based on body weight: blank control group (Group C), CUMS model group (Group H), FLX positive control group (FLX group), PC17 high dose group (PC17-H), PC17 medium dose group (PC17-M), and PC17 low dose group (PC17-L), with 10 mice in each group. The blank control group undergoes no stimulation, while the other groups replicate the CUMS model with continuous stimulation for 28 days, as shown in Table 3.

From the first day of modeling, all test substances are dissolved in normal saline. The FLX group is administered a dose of 10 mg/kg. The PC17 low dose, medium dose, and high dose groups are administered a concentration of 1×10⁸ CFU/mL, 1×10⁹ CFU/mL, and 1×10¹⁰ CFU/mL, respectively. The blank control and model groups are given normal saline. All groups receive daily oral gavage at a volume of 200 μL per mouse for four weeks.

CUMS stimulation consists of two stimuli per day, including one long-term and one short-term, with each stimulus applied at different times over a two-day period.

Long-term stimulation (24 h each): food and water deprivation, inclined cage, wet bedding, day-night reversal, and strobe light stimulation, for a duration of 24 h each.

Short-term stimulation: restraint for 4-6 h, hot water swimming for 10 min, ice water swimming for 10 min, tail suspension for 10 min, ice stimulation for 1 h, and Tail-pinch stimulation for 3 min.

The specific timeline for modeling is shown in Table 3.

TABLE 3
CUMS modeling stimulation schedule table

| Time | Day 1 | Day 2 | Day 3 | Day 4 | Day 5 | Day 6 | Day 7 |
|---|---|---|---|---|---|---|---|
| Week | tail | hot | ice water | tail | restraint + | ice | tail |
| 1 | pinch + | water | swimming + | suspension + | strobe | stimulation + | pinch + |
| | constant | swimming + | food | constant | light | water | wet |
| | darkness | inclined | deprivation | light | stimulation | deprivation | bedding |
| | | cage | | | | | |
| Week | ice water | tail | restraint + | tail | hot | ice | tail |
| 2 | swimming + | suspension + | inclined | pinch + | water | stimulation + | suspension + |
| | constant | strobe | cage | wet | swimming + | constant | food |
| | light | light | | bedding | water | darkness | deprivation |
| | | stimulation | | | deprivation | | |
| Week | restraint + | ice water | tail | hot | tail | restraint + | ice |
| 3 | water | swimming + | pinch + | water | suspension + | constant | stimulation + |
| | deprivation | inclined | strobe | swimming + | wet | light | food |
| | | cage | light | constant | bedding | | deprivation |
| | | | stimulation | darkness | | | |
| Week | hot | tail | ice water | tail | restraint + | ice | tail |
| 4 | water | suspension + | swimming + | pinch + | inclined | stimulation + | pinch + |
| | swimming + | strobe | constant | water | cage | constant | wet |
| | food | light | darkness | deprivation | | light | bedding |
| | deprivation | stimulation | | | | | |

After the experiment, mice are subjected to behavioral tests, including the sucrose preference test, tail suspension test, and forced swimming test. Serum samples are analyzed using ELISA kits. HE staining is performed on the mice's jejunal tissue, and the relative abundance of gut bacteria in the mice is determined.

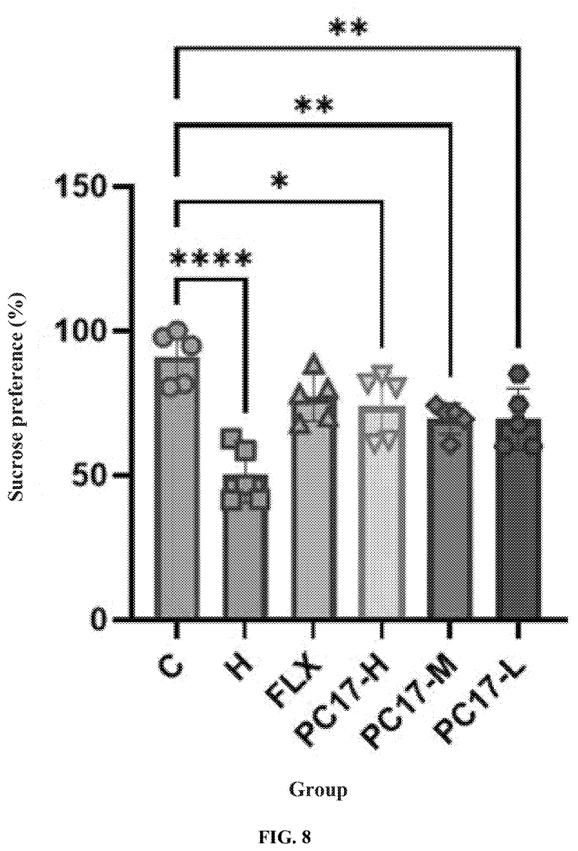

3) Experimental results: The results of the sucrose preference test are shown in FIG. 8. Anhedonia, a core symptom of depression, is assessed by the sucrose preference test to reflect the degree of anhedonia in model mice. Four weeks after modeling, the sucrose preference rate of mice in Group H is 50.23%, which is significantly lower than 91.00% in Group C (P<0.0001). Compared with Group H, administration of PC17 improves the sucrose preference rate to some extent. Specifically, high, medium, and low doses of PC17 increase the sucrose preference rate of model mice by 23.71%, 19.39%, and 19.20%, respectively.

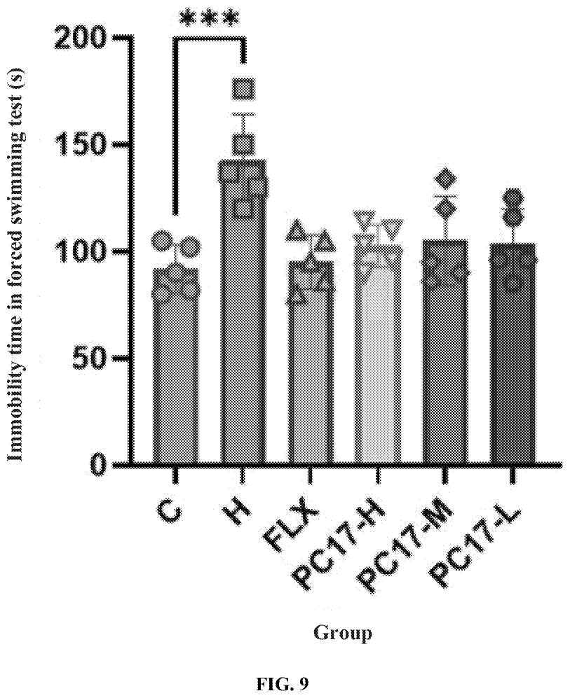

Immobility time in the forced swimming and tail suspension tests serves as an indicator of behavioral despair in mice. In the forced swimming test, as shown in FIG. 9, the average immobility time of mice in Group His 142.6 s, which is significantly longer than the 91.8 s in Group C (P<0.05). The immobility times of the FLX group, PC17-H group, PC17-M and PC17-L group are 95.2 s, 106.2 s, 105 s, and 103.6 s, respectively, showing no significant difference from Group C. This indicates that PC17 can effectively alleviate CUMS-induced despair behaviour, and the PC17 high dose exhibits an effect comparable to that of the positive control drug in the forced swimming test.

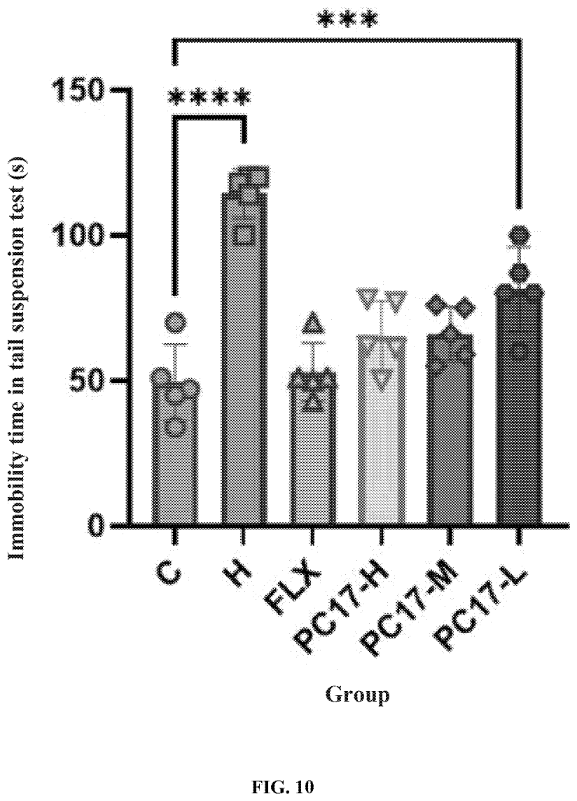

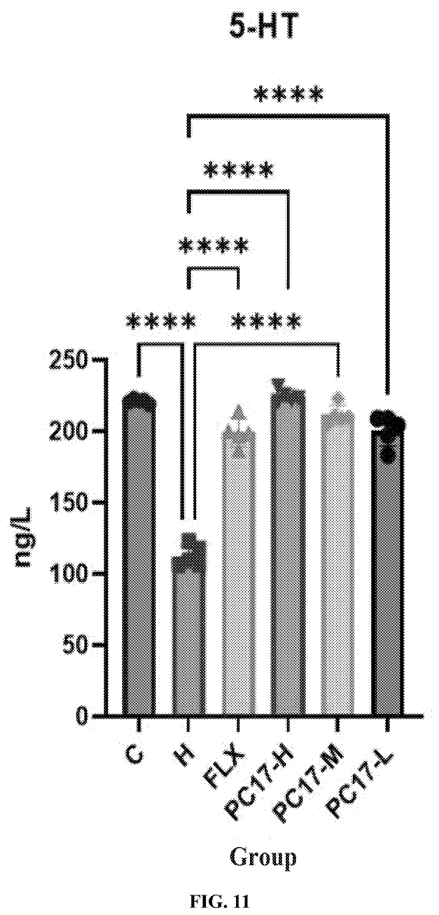

In the tail suspension test, as shown in FIG. 10, after 5 min of observation, the immobility time of mice in Group H is 114.4 s, significantly higher than the 49.4 s in Group C (P<0.0001), further confirming the effectiveness of CUMS modeling. The immobility time of the PC17-L group is 81.4 s, which is significantly different from that of Group C (P<0.001). The immobility times of the FLX group, PC17-H group, and PC17-M group are 53 s, 65.6 s, and 66.2 s, respectively, showing no significant difference from the blank control group. Compared with Group H, PC17-H and PC17-M reduce immobility time by approximately 42%, demonstrating excellent antidepressant effects in the tail suspension test.

5-HT is an important monoamine neurotransmitter that regulates emotional and cognitive functions. As shown in FIG. 11, in 5-HT levels, the serum 5-HT concentration in Group H (112.45 ng/L) is significantly lower than that in Group C (221.11 ng/L) (P<0.0001), which is consistent with the decreased serum 5-HT concentration observed in patients with clinical depression. Fluoxetine restores 5-HT to 199.12 ng/L by inhibiting 5-HT reuptake. Serum 5-HT levels in the PC17-H group (224.63 ng/L) and PC17-M group (211.52 ng/L) are not significantly different from those in Group C (221.11 ng/L), suggesting that high and medium doses of PC17 may achieve 5-HT homeostasis reconstruction through specific pathways.

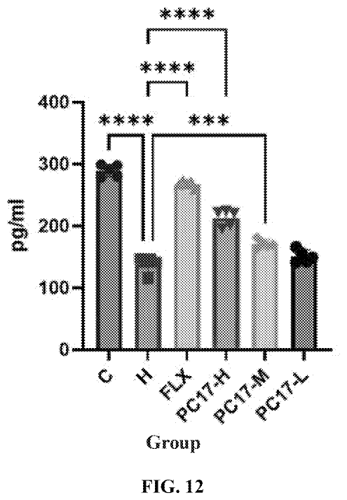

5-HTP is the precursor of 5-HT, and a decrease in its level may lead to reduced 5-HT synthesis, which further affects emotional regulation. As shown in FIG. 12, compared with the model group Group H (138.31 μg/mL), the 5-HTP levels in the FLX group, PC17-H group, and PC17-M group are significantly increased. Specifically, the 5-HTP levels in the PC17-H and PC17-M groups are 213.15 μg/mL and 171.04 μg/mL, respectively. The 5-HTP level in the PC17-L group (151.07 μg/mL) is not significantly different from that in Group H, although an upward trend is observed, the effect of PC17 on the restoration of 5-HTP levels in mice is dose-dependent.

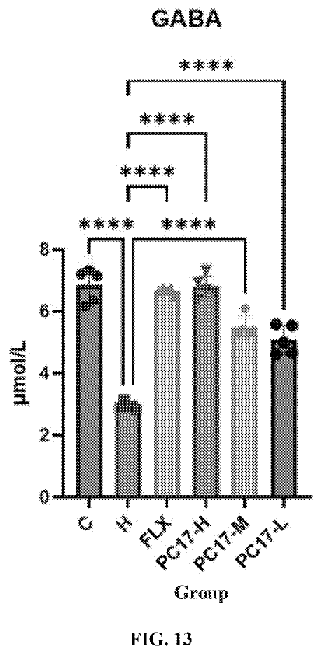

GABA is the main inhibitory neurotransmitter, and its levels are typically reduced in depression, which may lead to increased neuronal excitability. Regarding the GABAergic system, as shown in FIG. 13, compared with Group H (2.95 μmol/L), the serum 5-HT levels of mice in the FLX treatment group and the PC17 high, medium, and low dose groups are significantly restored (P<0.0001). The GABA levels in the FLX group and the PC17-H groups are 6.65 μmol/L and 6.81 μmol/L, respectively, approaching the 6.84 μmol/L in Group C, indicating that different doses of PC17 exert heterogeneous effects on the regulation of the GABA system.

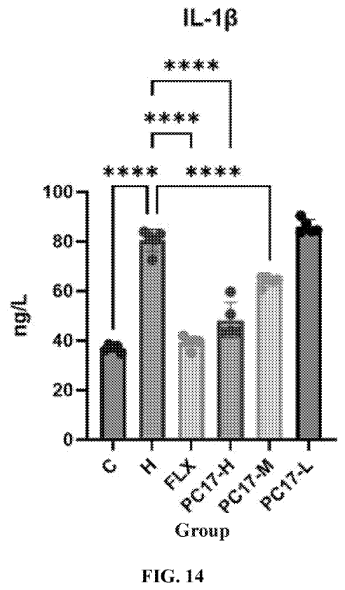

As a core mediator of central nervous system inflammation, excessive release of IL-1β can activate the NF-κB pathway in microglia, induce apoptosis of hippocampal neurons, and impair synaptic plasticity. As shown in FIG. 14, compared with Group C (36.95 ng/L), the serum IL-1ß level of mice in Group H (80.49 ng/L) is significantly increased by 117.8%. Following intervention with high and medium doses of PC17, the serum IL-1β level is decreased by 51.2% and 40.1%, respectively (P<0.0001).

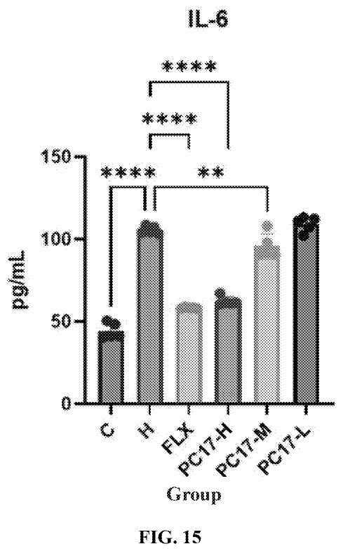

IL-6 regulates neurogenesis and synaptic remodeling through the JAK-STAT signaling pathway. As shown in FIG. 15, compared with Group C (44.32 μg/mL), the serum IL-6 level of mice in Group H is significantly increased by 138.1%, reaching 105.53 μg/mL. The effect of PC17 on serum IL-6 is dose-dependent. The PC17-H group significantly reduces IL-6 to 62.27 μg/mL (P<0.0001), and the PC17-M group significantly reduces IL-6 to 95.75 μg/mL (P<0.01). However, no significant difference is observed in the effect of the low dose of PC17 on serum IL-6. This indicates that a threshold concentration is required for PC17 to exert anti-inflammatory effects through IL-6 reduction.

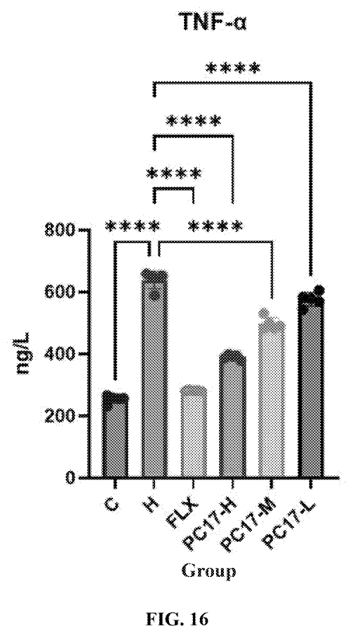

TNF-α is a key factor in the regulation of blood-brain barrier permeability, and its overexpression can promote the infiltration of peripheral immune cells. As shown in FIG. 16, the regulation of TNF-α by PC17 in each dose group also exhibits dose-dependent inhibitory characteristics. Compared with interleukins, CUMS mice show a greater response to TNF-α, with serum TNF-α of mice in Group H increasing from 254.18 ng/L in Group C to 639.29 ng/L (P<0.0001). Compared with Group H, PC17 intervention demonstrates a gradient regulatory effect, with serum TNF-α levels in the PC17-H group (390.68 ng/L), PC17-M group (497.46 ng/L), and PC17-L group (576.35 ng/L) being significantly decreased (P<0.0001).

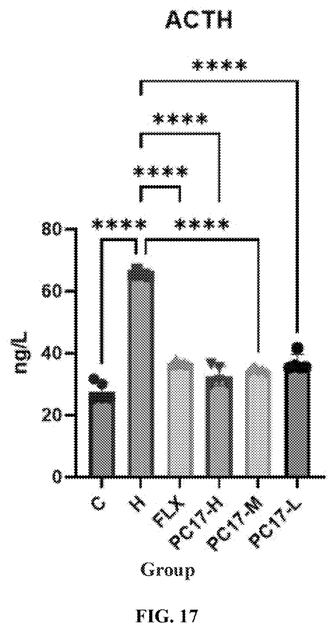

As shown in FIG. 17, compared with Group C (27.59 ng/L), the serum ACTH level of mice in Group H (65.57 ng/L) is significantly increased. Compared with Group H, the serum ACTH levels in the FLX group (36.70 ng/L), PC17-H group (32.61 ng/L), PC17-M group (34.29 ng/L), and PC17-L group (36.78 ng/L) are significantly decreased (P<0.0001), although they do not restore to the level observed in Group C. PC17 intervention significantly reverses CUMS-induced ACTH overexpression.

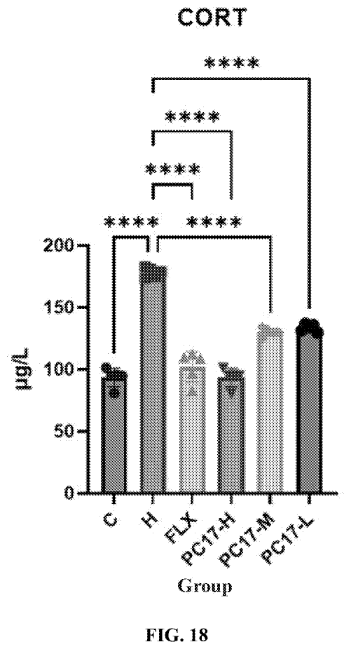

As shown in FIG. 18, compared with Group C (93.47 μg/L), the serum CORT level of mice in Group H (178.32 μg/L) is significantly increased (P<0.0001). Compared with Group H, the serum CORT levels in the PC17-M group (130.20 μg/L) and PC17-L group (133.60 μg/L) are significantly decreased (P<0.0001). Although they do not restore to the level of Group C, they effectively reduce serum CORT levels in CUMS mice. The PC17-H group (93.47 μg/L) and FLX group (102.00 μg/L) achieve levels close to those of Group C.

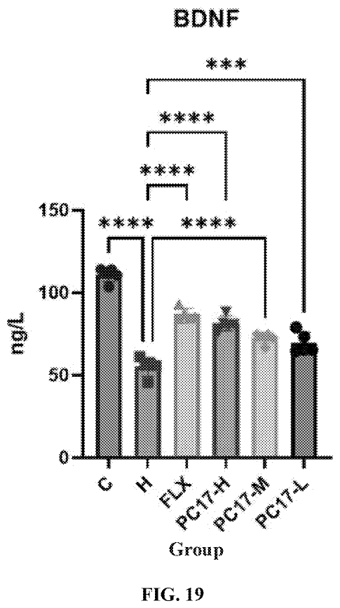

As shown in FIG. 19, compared with Group C (110.92 ng/L), the serum BDNF level of mice in Group H (55.12 ng/L) subjected to CUMS stimulation is significantly decreased by 50.3% (P<0.0001), which is consistent with the downward trend in BDNF protein expression observed in patients with clinical depression. Compared with Group H, the BDNF levels in the FLX group (86.88 ng/L), PC17-H group (81.42 ng/L), PC17-M group (72.57 ng/L), and PC17-L group (69.56 ng/L) are significantly increased, although they fail to restore to Group C levels.

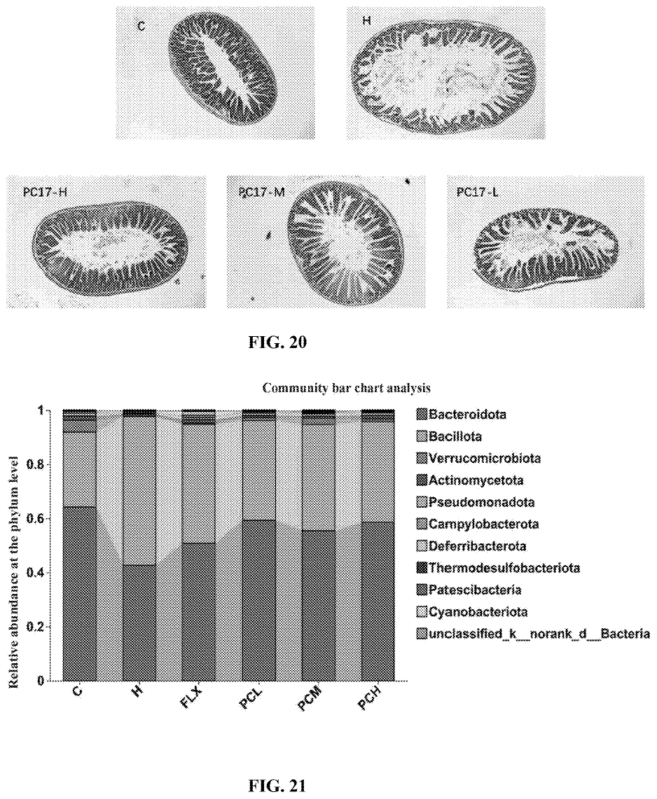

As shown in the HE staining results of mouse jejunum tissue in FIG. 20, the jejunal villi in Group H of the CUMS model group are significantly shortened and fused, with expanded and deformed crypts. The villus height-to-crypt depth (V/C) ratio is significantly reduced, indicating impaired absorptive function. Extensive neutrophil and lymphocyte infiltration is observed in the lamina propria, along with local erosion, a decrease in goblet cell numbers, and the absence of a mucus layer. In mice administered with lactic acid bacteria PC17, the inflammatory state is alleviated to different degrees. The V/C ratio approaches normal levels, goblet cell density is restored to near normal, and colonic tissue morphology is partially recovered.

Based on 16S rRNA gene sequencing analysis, as shown in FIG. 21, the composition of bacterial phyla is analyzed among different groups at the phylum level. The abundance of Bacteroidota in the CUMS stimulation group is significantly lower than that in Group C, with a highly significant statistical difference observed between Group C (64.16%) and Group H (42.69%) (P<0.001). A significant difference is also found between the FLX group (50.80%) and Group H (P<0.05). Following PC17 intervention, the abundance of Bacteroidota is restored to 59.29% in the PC17-L group, 55.45% in the PC17-M group, and 58.54% in the PC17-H group, indicating that PC17 reverses the CUMS-induced reduction in Bacteroidota abundance.

The proportion of Bacillota in Group C is 27.80%. After intervention with low, medium, and high doses of PC17, the Bacillota proportion in the PC17-L (36.78%), PC17-M (39.25%), and PC17-H (37.27%) groups is decreased by 18.11%, 15.64%, and 17.62%, respectively. Compared to Group H. FLX intervention significantly reduces the Bacillota proportion by 10.95% (P<0.01). PC17 intervention significantly increases the abundance of Verrucomicrobiota, which is reduced by CUMS stimulation. CUMS stimulation also leads to a significant increase in Actinomycetota in the intestine.

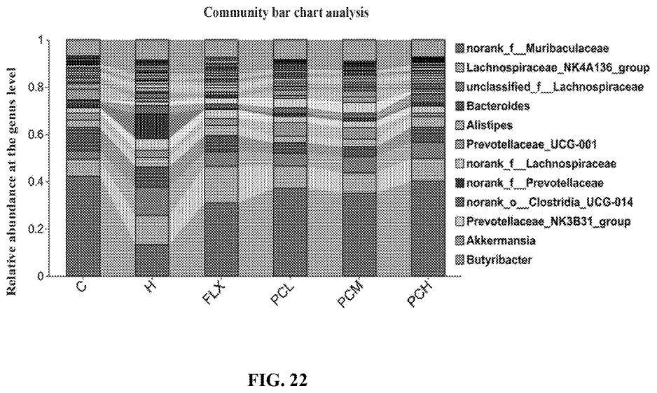

As shown in FIG. 22, the composition of bacterial genera is analyzed among different groups at the genus level. Group C is characterized by a predominance of norank_f_Muribaculaceae, Lachnospiraceae_NK4A136_group, and Bacteroides, forming a distinct genus abundance structure. In Group H, norank_f_Muribaculaceae is significantly decreased by 30.02% compared to Group C, while the level of unclassified_f_Lachnospiraceae is significantly increased. Relative to CUMS-stressed mice, PC17 administration significantly upregulates norank_f_Muribaculaceae and downregulates unclassified f Lachnospiraceae, making their levels close to those observed in Group C. The hierarchical distribution of high-abundance genera, including norank_f_Muribaculaceae, Lachnospiraceae_NK4A136_group, and Prevotellaceae_NK3B31_group, in the PC17-H group most closely aligned with that of Group C. Based on genus-level abundance structure, the genus composition of the PC17-H group is the closest to that of Group C, indicating a relatively better therapeutic effect. In contrast, the PC17-L and PC17-M groups exhibit more significant differences from Group C in terms of genus abundance ratios and structure, showing a comparatively lesser effect.

The foregoing description is not intended to limit the scope of the present disclosure, and the disclosure is not restricted to the embodiments described above. Any modifications, alterations, additions or substitutions made by a person skilled in the art within the spirit and scope of the present disclosure shall also fall within the scope of the present disclosure.

What is claimed is:

1. A use for a probiotic strain with efficacy in improving depression-like symptoms in a preparation for antidepressant drugs, the probiotic strain is Limosilactobacillus fermentum PC17 strain, with a culture collection number CGMCC No. 33866.

2. A use for a probiotic microecological preparation with efficacy in improving depression-like symptoms in a preparation for antidepressant drugs, comprising Limosilactobacillus fermentum PC17, the strain having a culture collection number CGMCC No. 33866, wherein the preparation reduces serum levels of IL-1B, IL-6, TNF-α, CORT, and ACTH, and increases serum levels of GABA and BDNF.

3. The use according to claim 2, wherein an effective viable cell count of Limosilactobacillus fermentum PC17 in the probiotic microecological preparation is greater than or equal to 1×10⁷ CFU/mL.

4. The use according to claim 2, wherein a method for preparing the probiotic microecological preparation comprising the following steps: inoculating Limosilactobacillus fermentum PC17, deposited under the culture collection number CGMCC No. 33866, into a fermentation medium at a volume ratio of 1-3%, and culturing at 35-40° C. for 15-20 h to obtain a lactic acid bacterial culture, centrifuging the obtained lactic acid bacterial culture to obtain a lactic acid bacterial pellet, adjusting a bacterial concentration to 1×10⁷-1×10⁹ CFU/mL using a sterile PBS; adding a lyoprotectant in an amount equal to the volume of the sterile PBS and mixing uniformly, then placing the mixture in a refrigerator at −80° C. overnight for pre-freezing, and freeze-drying the pre-frozen sample to obtain the probiotic microecological preparation.

5. The use according to claim 4, wherein a formula of the lyoprotectant is 118.2 g/L trehalose, 17.1 g/L L-cysteine, 10.3 g/L sorbitol, 1.7 g/L sodium acetate and 120 g/L skimmed milk powder.
